(19) United States
(12) Patent Application Publication
(10) Pub. No.: US 2007/0134684 A1
(43) Pub. Date: Jun. 14, 2007

(54) USE OF ADENINE AS A METHOD FOR CONTROLLED IMMOBILIZATION OF NUCLEIC ACIDS AND THEIR ANALOGS ON GOLD SURFACES

(76) Inventors: Dmitri Y. Petrovykh, Alexandria, VA (US); Lloyd J. Whitman, Alexandria, VA (US); Michael J. Tarlov, Bethesda, MD (US); Aric M. Opdahl, La Crosse, WI (US); Hiromi Kimura-Suda, Tokyo (JP)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(21) Appl. No.: 11/457,713

(22) Filed: Jul. 14, 2006

Related U.S. Application Data
(60) Provisional application No. 60/699,488, filed on Jul. 15, 2005.

(51) Int. Cl. C12Q 1/68; C12M 1/34; B05D 3/00

(52) U.S. Cl. 435/6; 435/287.2; 427/2.11

(57) ABSTRACT

The method provides for attaching nucleic acids to a surface at a controlled grafting density in a controlled conformation by contacting an immobilization solution of nucleic acids containing at least one block of adenine nucleotides to a surface for a sufficient period of time to allow attachment to the surface. Another aspect of the methods described provides for controlling the grafting density of immobilized oligonucleotides by coadsorption/displacement by oligo(dA). Another aspect provides for a method of immobilizing oligonucleotides in complex conformations by varying the number and position of the block(s) of adenine nucleotides in the sequence of said oligonucleotides. Another aspect provides for controlled immobilization of a functional unit, such as a ligand, a molecule, a macromolecule, an aptamer, a lectin, an immunoglobulin, an antibody, a biomolecule, a solid state particle, a vesicle, or a label to a surface via attachment to at least one block of adenine nucleotides.

### CROSS-REFERENCE TO RELATED APPLICATIONS

This Application is a Non-Prov of Prov (35 USC 119(e)) application 60/699488 filed on Jul. 15, 2005, the entirety of which is hereby incorporated by reference.

### BACKGROUND OF THE INVENTION

Gold has long been a substrate of choice for the attachment of organic and biological molecules to solid surfaces [Love et al., Self-Assembled Monolayers of Thiolates on Metals as a Form of Nanotechnology, Chem. Rev. (Washington, DC) 105, 1103-69 (2005)]. In particular, there are a variety of applications in nanotechnology and biotechnology where single-stranded DNA (ssDNA) is chemically attached to a gold surface in order to utilize the capability of ssDNA for specific molecular recognition. In these and many other applications, where DNA is used as a molecular recognition element or a structural element, it is important to reliably control the surface density and molecular conformation of the DNA. (DNA attached to a surface by any means is hereafter referred to as “immobilized” DNA.) One common method to immobilize ssDNA on gold is to modify the DNA with a terminal thiol group and then link the DNA to the surface with a sulfur-gold bond. The spacing and conformation of ssDNA molecules immobilized in this way can be further modified after deposition by exposing the surface to a competing small organic thiol, e.g., 6-mercapto-1-hexanol (MCH), 11-mercapto-1-undecanol (MCU), etc. [Herne and Tarlov, Characterization of DNA Probes Immobilized on Gold Surfaces, J. Am. Chem. Soc. 119, 8916-20 (1997)] The use of thiol chemistry in conjunction with gold surfaces, however, has many practical disadvantages, and thus few commercial applications have successfully adopted thiol linkers for immobilization of DNA.

Surfaces functionalized by immobilization of ssDNA are the basic component of DNA sensors and hybridization microarrays used for genetic analysis, biosensor applications, and biomedical research [Tarlov and Steel, DNA-Based Sensors in Biomolecular Films: Design, Function, and Applications. 545-608 (Marcel Dekker, Inc., New York. 2003)]. There is also widespread interest in using DNA as a structural and a molecular recognition component in nanotechnology, for example, for tethering nanoscale objects to each other and to surfaces. One desirable attribute of a surface functionalized with ssDNA is efficient and reproducible hybridization with target ssDNA in solution, the “target” having a nucleotide sequence at least in part complementary to the immobilized (or “probe”) sequence. Model studies of DNA oligonucleotides (oligos) attached to gold via thiol linkers suggest that efficient hybridization occurs when the spacing between DNA probe strands is comparable to the length of the strand, and when the probes have an upright orientation with respect to the surface (i.e., oriented roughly perpendicular to the surface or extending away from the surface) [Gong et al., Hybridization Behavior of Mixed DNA/Alkylthiol Monolayers on Gold: Characterization by Surface Plasmon Resonance and P-32 Radiometric Assay, Anal. Chem. 78, 3326-34 (2006)]. Single-stranded DNA films possessing both of these qualities are, unfortunately, difficult to prepare in a robust and reproducible fashion. For example, when the lateral spacing between nearest-neighbor ssDNA strands is large, nucleobases within the strands are often observed to interact with the gold causing the ssDNA to “lie flat” or even chemisorb on the surface [Petrovykh et al., Quantitative Analysis and Characterization of DNA Immobilized on Gold, J. Am. Chem. Soc. 125, 5219-26 (2003)].

Thiol-gold attachment chemistry is a common method for immobilizing organic and biomolecules on gold surfaces [Luderer and Walschus, Immobilization of Oligonucleoticles for Biochemical Sensing by Self-Assembled Monolayers: Thiol-Organic Bonding on Gold and Silanization on Silica Surfaces in Immobilisation of DNA on Chips 1, 37-56 (2005)]. The commercial availability of thiol-modified ssDNA also encourages the use of this method in research and development. At least three disadvantages limit the use of this attachment method. One disadvantage is the instability of the thiol-gold bond solutions at elevated temperatures, e.g., many standard DNA hybridization protocols call for solution temperatures as high as 90° C. This instability can in part be alleviated by using anchoring linkers with multiple thiol groups. However, such modifications are not commercially available and therefore require custom synthesis. A second disadvantage is the inability to control neither the conformation of the immobilized ssDNA molecules nor their grafting density. A post-immobilization exposure to MCH, MCU, or other short-chain organic water-soluble thiols is widely used to reduce the surface density and promote an upright conformation, and to thereby increase the hybridization efficiency [see, for example, Herne and Tarlov, Characterization of DNA Probes Immobilized on Gold Surfaces, J. Am. Chem. Soc. 119, 8916-20 (1997) and Lee et al., Surface Coverage and Structure of Mixed DNA/Alkylthiol Monolayers on Gold: Characterization by XPS, NEXAFS, and Fluorescence Intensity Measurements, Anal. Chem. 78, 3316-25 (2006)]. However. for some applications a high concentration of highly chemically reactive small organic thiols may cause contamination problems. Contamination is a third disadvantage of thiol-gold chemistry, because contaminants similar to MCH are often unintentionally introduced during the preparation of thiol-modified DNA [Lee et al., Evidence of Impurities in Thiolated Single-Stranded DNA Oligomers and Their Effect on DNA Self-Assembly on Gold, Langmuir 21, 5134-41 (2005)]. These and other practical disadvantages have limited the commercial use of DNA immobilization via thiol-gold chemistry to relatively simple applications, such as the functionalization of gold nanoparticles.

It is common practice to use a nonreactive linker between the thiol anchoring group and the specific nucleotide sequence of the probe DNA. The purpose of such linkers, which we will refer to as “vertical” spacers, is to separate the specific sequence a distance away from the surface in order to make it more sterically available for hybridization with a complementary target. Common vertical spacers include simple organic chains [e.g., alkanes. poly(ethylene glycol), etc.] and homo-oligonucliodies [particularly, oligo(dA) or thymine (oligo(dT)]. Typical vertical spacers are between about 3 and about 15 monomer units long. Although there has been anecdotal evidence from a variety of experiments with DNA and gold surfaces that oligo(dA) spacers may, in fact, interact with gold surfaces, the results have usually been described as unexpected and were not explained. [see, for example. Anne et al., 3′-Ferrocene-Labeled Oligonucleotide Chains End-Tethered to Gold Electrode Surfaces: Novel Model Systems for Exploring Flexibility of Short DNA Using Cyclic Voltammetry, J. Am. Chem. Soc. 125, 1112-3 (2003)]. The only application that has specifically called for the use of oligo(dA) spacers for ssDNA probe immobilization is functionalization of gold nanoparticles, where a few empirically derived recipes call for oligo(dA) vertical spacers on thiolated ssDNA. Such functionalization of gold nanoparticles has been systematically described in Storhoffet al., Sequence-Dependent Stability of DNA-Modified Gold Nanoparticles, Langmuir 18, 6666-70 (2002).

Mirkin et al. U.S. Pat. No. 6,903,207 provides for methods of preparing stable nanoparticle-oligonucleotide complexes using a two-step process, which begins with immobilization in water and continues in salt buffer (of constant or variable ionic strength). Note that although Mirkin et al. have made observations about adenine oligonucleotides binding to gold, they did not recognize the potential advantages of these observations, and they did not teach how these observations can be applied to control DNA density with or without a coupling moiety such as sulfur. Mirkin states that the oligonucleotides have a spacer portion and a recognition portion, and that the spacer portion is designed so that it is bound to the nanoparticle. The spacer portion is described as having a moiety covalently bound to it, that is, Mirkin explicitly refers to a type of oligonucleotide functionalization (e.g., with a thiol). Mirkin discusses that as a result of the binding of the spacer portion of the recognition nucleotide to the nanoparticles, the recognition portion is spaced away from the surface of the nanoparticles. This definition of a spacer sequence in an oligonucleotide probe is consistent with the general use of the term in the literature, whereby the implied spacing is away from the surface in question. Mirkin's type of spacing can be considered vertical spacing rather than lateral spacing, where the latter is specifically designed to control the lateral distance between adjacent oligonucleotides on a surface. Similarly, Mirkin et al. describe using “diluent oligonucleotides” to control the surface density of immobilized ssDNA probes, but the method they describe is a variation of the MCH dilution method, whereby the diluent oligonucleotides statistically reduce the surface density of the immobilized ssDNA probes via a surface crowding effect. The diluent oligos are also specifically assumed to interact with the surface only via a functional moiety and to have a sequence not complementary to the recognition portion of the probe oligos.

Thus there is a need in the art for a method for immobilizing oligonucleotides to a surface where the surface density, conformation, and relative placement of oligonucleotides on the surface can be controlled, and where the method does not require thiol modification of the DNA or post-immobilization exposure to small organic thiols. These and other needs are addressed by the present invention.

### BRIEF SUMMARY OF THE INVENTION

The methods provide for attaching nucleic acids to a surface at a controlled surface density (grafting density) in a controlled conformation comprises contacting an immobilization solution, comprising nucleic acids containing at least one block of adenine nucleotides, to a surface for a sufficient period of time and under appropriate conditions to allow attachment to the surface. Another aspect of the methods described further provides for controlling the surface density (grafting density) of immobilized oligonucleotides by coadsorption with and/or displacement by oligo(dA). Another aspect further provides for a method of immobilizing oligonucleotides in complex conformations by varying the number and position of the block(s) of adenine nucleotides in the sequence of said oligonucleotides. Another aspect further provides for immobilizing other functional units, such as a ligand, a molecule, a macromolecule, an aptamer, a lectin, an immunoglobulin, an antibody, a biomolecule, a solid state particle, a vesicle, or a label to a surface via linkage to at least one block of adenine nucleotides.

### BRIEF DESCRIPTION OF THE DRAWINGS

FIG. 1 is a graph showing competitive adsorption of oligo(dA) on gold;

FIG. 2 is an idealized schematic of d(Tₘ-Aₙ) oligonucleotides adsorbed on gold;

FIG. 3 shows data confirming the L-shaped conformation of a model oligo;

FIG. 4 shows data indicating the effect of deposition conditions on conformation:

FIG. 5 is a chart showing DNA coverage (grafting density) for d(Tₘ-Aₙ) oligonucleotides adsorbed on gold:

FIG. 6 is representation of reversible hybridization;

FIG. 7 is representation showing control of grafting density of d(Tₘ-Aₙ) using coadsorption/displacement with oligo(dA);

FIG. 8 is representation of d(Tₘ-Aₙ₋Tₘ) adsorbed on gold in “U” and “J” conformations;

FIG. 9a is a representation of d(Tₘ-Aₙ₋Tₘ-Aₙ₋Tₘ) adsorbed on gold in “W” conformation;

FIG. 9b is a representation of d(Aₙ₋Tₘ-Aₙ) adsorbed on gold in “Ω” conformation;

FIG. 10 shows FTIR spectra obtained from ssDNA on gold;

FIG. 11 shows XPS spectra for ssDNA on gold:

FIG. 12 shows FTIR spectra and XPS oligonucleotide coverages before and after denaturing;

FIG. 13 shows the FTIR spectra of d(T₂₅-A₂₀) film;

FIG. 14 shows data of Adenine nucleotide coverages;

FIG. 15 shows FTIR spectra from a d(T₂₅-A₅) oligo film.

### DETAILED DESCRIPTION OF THE INVENTION

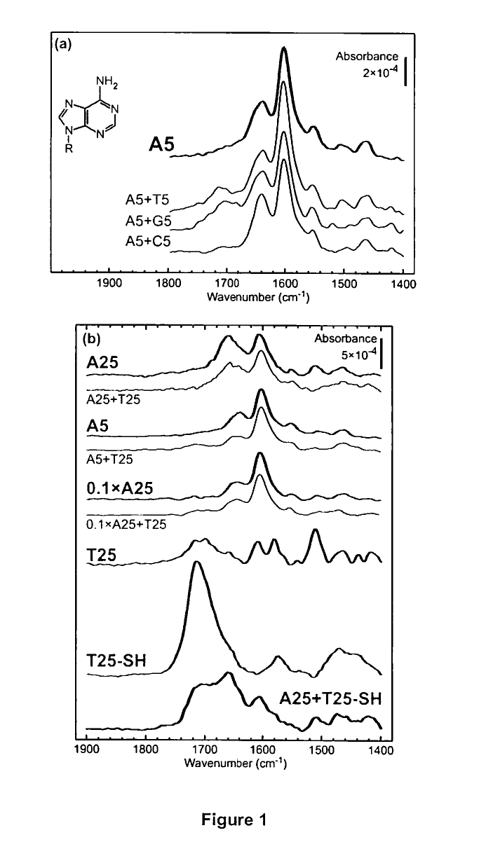

Provided is a method for immobilizing DNA to gold, which takes advantage of the high intrinsic affinity of adenine nucleotides (dA) for gold, as reported by Kimura-Suda et al., Base-Dependent Competitive Adsorption of Single-Stranded DNA on Gold, J. Am. Chem. Soc. 125, 9014-5 (2003), incorporated herein by reference in full. When a gold surface is exposed to a solution with a mixture of different oligonucleotides, oligo(dA), if present in that mixture, adsorbs on gold almost exclusively. FIG. 1 presents data indicating that, under a wide range of experimental conditions, blocks of oligo(dA) will attach to gold surfaces, even in the presence of competition from other nucleotides, hybridization, and other adsorbing species that can be present in practical immobilization solutions.

Under appropriate deposition conditions, blocks of n adenine DNA nucleotides, denoted d(Aₙ) or d(A), present in solution either as constituents of longer nucleotide sequences or as separate oligonucleotides, will preferentially adsorb on gold, displace other adsorbed oligonucleotides, and prevent subsequent adsorption of other oligonucleotides. The surface density, conformation, and relative placement of DNA molecules on gold can be controlled by adjusting the length n of the d(Aₙ) blocks and/or the immobilization conditions. Because the high affinity for gold is the property of adenine nucleobases, the approach can be readily extended from DNA oligonucleotides to RNA (some of which naturally contain polyA sequences), PNA, and any other natural or synthetic nucleic acids or their analogs that contain adenine.

The method provided for attaching nucleic acids or nucleic acid analogs to a surface at a controlled surface density (grafting density) in a controlled conformation comprises contacting an immobilization solution, comprising nucleic acids or nucleic acid analogs containing at least one block of adenine nucleotides or adenine nucleotide analogs, to a surface for a sufficient period of time and under appropriate conditions to allow attachment to the surface. Nucleic acid analogs are defined herein to include both natural and synthetic analogs of nucleic acids. Adenine nucleotide analogs are defined to include adenine-containing monomer constituents of nucleic acid analogs. The surface can be gold, iron, cobalt, nickel, copper, ruthenium, rhodium, palladium, silver, osmium, iridium, platinum or alloys thereof [interactions of organic and biomolecules with such metal surfaces are discussed, for example, in Love et al., Self-Assembled Monolayers of Thiolates on Metals as a Form of Nanotechnology, Chem. Rev. (Washington, DC) 105, 1103-69 (2005); Giese and McNaughton, Surface-Enhanced Raman Spectroscopic and Density Functional Theory Study of Adenine Adsorption to Silver Surfaces, J. Phys. Chem. 106. 101-12 (2002); Chen et al., Self-Assembly of Adenine on Cu(110) Surfaces, Langmuir 18, 3219-25 (2002)]. Most preferably, the surface is gold.

Solutions for DNA immobilization (DNA immobilization solutions) are well known in the art. A buffered aqueous solution of DNA for immobilization onto a surface typically includes at least three components: DNA, salt, and a buffering agent. Immobilization solution that does not contain DNA (i.e., before DNA is added, or as used for intermediate rinsing steps) is commonly referred to as an “immobilization buffer.” A person skilled in the art would know that there are many commercially-available immobilization buffers as well as many possible combinations of chemical compounds that can be used in immobilization buffers. In addition, most buffered solutions used for DNA hybridization (“hybridization buffers”) can also be used as immobilization buffers. It is also possible to create an immobilization buffer based on one or more non-aqueous solvent(s). The only requirements are that both DNA and salt are soluble in such a solvent (solvent combination) within the concentration limits. This requirement, however, tends not to be satisfied for organic solvents, which, unlike water, typically act as good solvents for either cations or anions, but not both.

In general, the effects of all the components of an immobilization solution on DNA immobilization are strongly interdependent, and accordingly the overall performance of a specific immobilization solution has to be considered on the basis of the combined effect of all the solution components, rather than as a simple function of any single solution component.

First, the concentration of ssDNA oligonucleotides (oligos) should be considered. The lowest limit of the concentration of oligonucleotides is determined by the combination of the desired surface density of the immobilized oligos, the surface area to be functionalized with DNA, and the volume of the DNA immobilization solution used in the specific immobilization. Specifically, the product of DNA concentration and solution volume has to be greater or equal to the product of surface density and surface area, i.e., the solution used to immobilize DNA within a given area has to contain enough DNA molecules to achieve the desired surface density within that area. A practical upper limit of the concentration of DNA oligonucleotides is determined in each case by the cost and availability of the respective oligos and the time available to carry out the immobilization. For example, purified ssDNA samples obtained from vendors are typically reconstituted to make a stock solution of 100-200 μM concentration, which effectively defines the upper limit for any subsequent use. The appropriate dilution of that initial stock solution is determined by considering the diffusion of DNA throughout the immobilization volume within the time allotted [Sheehan and Whitman, Detection Limits for Nanoscale Biosensors, Nano Letters 5, 803-7 (2005)].

For immobilization methods that require solution volumes >1 mL, DNA concentrations as low as 1 nM are commonly used. The highest limit of DNA concentration in this case is determined by the total amount of DNA available for the procedure and is typically <10 μM. For immobilization methods that use solution volumes between 1 nL and 1 μL (e.g., droplet “spotting” or printing methods), DNA concentrations are typically higher than those used in larger volumes—concentrations in the 10-100 μM range are not uncommon. The theoretical upper limit in all the above cases is determined by the solubility of ssDNA under the specific conditions. While reaching that limit in applications described in [0017] would typically be prohibitively expensive for the small volume applications described above, the concentration of ssDNA, in fact, can ultimately be limited by its solubility.

The density and conformation of the immobilized ssDNA are affected by both the chemical composition and the concentration (or ionic strength) of one or more salts added to the DNA immobilization solution. Single-stranded DNA is a strong polyelectrolyte. Oligonucleotides are negatively charged in aqueous solutions close to neutral pH and consequently experience mutual electrostatic repulsion. If no salt is added to the immobilization solution, this repulsion will limit the adsorption of DNA to a coverage equivalent to a small fraction of a close-packed film-potentially too small a coverage for practical applications and an adsorption regime where neither the surface density nor the conformation of the immobilized ssDNA can be reliably controlled. Salts that are strong electrolytes provide the most effective electrostatic screening and thus have the most pronounced effect on DNA immobilization.

Traditionally, DNA immobilization solutions contain physiological electrolytes, i.e., electrolytes that are common in physiological environments: sodium, potassium, calcium, magnesium, chloride, phosphate, and bicarbonate. Electrolyte anions do not strongly associate with ssDNA in aqueous solutions at neutral pH and therefore the choice of the anion(s) has a smaller effect on DNA immobilization that the choice of the cation(s). In practical use, the choice of anions is limited by their potential adverse effects as buffering agents (e.g., excessively shifting the solution pH) or affinity for metal surfaces. Chloride, phosphate, and bicarbonate anions are not believed to produce such adverse effects and therefore are commonly used.

Electrolyte cations, in contrast to anions, can and do strongly associate with ssDNA in aqueous solutions under most solution pH conditions where DNA remains stable (pH between 2 and 10) [Saenger, Principles of Nucleic Acid Structure (Springer-Verlag, New York, 1984); Bloomfield et al., Nucleic Acids: Structures, Properties, and Functions (University Science Books, Sausalito, Calif., 2000)]. Accordingly, the cations present in the DNA immobilization solution can dramatically affect the characteristics of the oligonucleotides, both those in solution and those adsorbed on the surface. Monovalent cations are most commonly used in immobilization buffers, in part because they are not generally considered likely to produce adverse effects during the subsequent DNA hybridization step(s).

The efficiency of ssDNA immobilization monotonically increases with increasing concentration of monovalent cations. Accordingly, using immobilization buffers with high ionic strength salts of monovalent cations (>1 M) is beneficial for increasing the rate of ssDNA immobilization (decreasing the time required for immobilization) and for increasing the final (or saturation) surface density of the immobilized ssDNA [Petrovykh et al., Quantitative Analysis and Characterization of DNA Immobilized on Gold. J. Am. Chem. Soc. 125, 5219-26 (2003)].

In contrast to the generally beneficial effect of monovalent cations, the effect of increasing the concentration of multivalent cations on the efficiency of ssDNA immobilization can be strongly non-linear [Petrovykh et al., Quantitative Analysis and Characterization of DNA Immobilized on Gold, J. Am. Chem. Soc. 125, 5219-26 (2003), Rant et al., Excessive Counterion Condensation on Immobilized ssDNA in Solutions of High Ionic Strength, Biophys. J. 85, 3858-64 (2003)]. Depending on the DNA sequence and on the pH, temperature, and composition of the immobilization solution, above some critical ionic strength of a multivalent cation salt, DNA can begin to agglomerate in solution [see, for example, Muntean et al., Influence of Ca²⁺ Cations on Low ph-Induced DNA Structural Transitions, Biopolymers 67, 282-4 (2002)]. Such DNA agglomerates will eventually form a layer or residue on any surface exposed to the respective solution—a form of DNA immobilization considered detrimental in practical applications.

The lowest limit of ionic strength for an immobilization solution is typically considered to be approximately 1-10 mM, depending on the specific salt(s) and other solution components used. Immobilization solutions with ionic strengths significantly below the 1-10 mM range can result in inconsistent, unpredictable, and poorly controlled DNA immobilization, due in part to the strong effect of any contaminants that are typically present in immobilization solutions at comparable concentrations.

For most applications, the practical lowest limit of ionic strength for an immobilization solution is equal to the effective ionic strength of its ssDNA component, i.e., the solution should contain at least as many cations as are necessary to neutralize the poly-anionic ssDNA under the specific pH, temperature, and other bulk parameters of the specific immobilization solution.

A person skilled in the art would understand that all but extremely meticulously prepared ssDNA samples will be likely to have some amount of residual salt(s) or counterions associated with them. That residual amount of associated salt or counterions dissolved in the volume of an immobilization solution represents the lowest physically possible ionic strength of the salt component in that immobilization solution.

Buffering agents are added to stabilize the pH of a DNA immobilization solution. Solution pH changes between 2 and 10 are generally considered safe for maintaining the integrity of synthetic oligonucleotides. For ssDNA samples derived directly from natural sources, e.g., cell lysate, the pH range of stability is typically narrower, to the extent determined by the amount of residual damage accumulated during enzymatic digestion and other pre-processing steps.

The primary effect of changing the pH of a DNA immobilization solution is changing the effective charge of poly-anionic ssDNA. The solution pH also directly or indirectly affects the properties of other solution components. Unbuffered solutions, in principle, can be used to immobilize ssDNA on surfaces, but can result in inconsistent, unpredictable, and poorly controlled DNA immobilization, because in an unbuffered solution, pH near a surface can be arbitrarily different from the pH of the bulk solution. Some salts used in DNA immobilization solutions can act as buffering agents. For example, monopotassium phosphate (KH₂PO₄) is commonly used in DNA immobilization and hybridization buffers in part because it acts as a weak buffering agent near neutral pH.

One skilled in the art would understand that a wide variety of organic and inorganic buffering agents can be used in a DNA immobilization solution. Customized buffering agents are routinely prepared by individual practitioners and can also be obtained from any number of commercial sources.

A wide variety of organic and inorganic compounds are commonly introduced as additives, at concentrations comparable to or less than those of the components described above, into DNA immobilization solutions. Some such compounds have well-known effects and are readily available both commercially and through simple custom synthesis. For example, chelators, such as the commonly-used ethylenediaminetetraacetic acid (EDTA), are a particularly useful class of additives for DNA immobilization solutions, because they help to suppress the potentially adverse effect of multivalent counterions (particularly tri- and tetravalent counterions) that are often present as contaminants in DNA immobilization solutions. Any compounds added to a DNA immobilization solution should not be excessively corrosive for the chosen surface material. Compounds added to a DNA immobilization solution should not have a high enough affinity for the chosen substrate material and/or should not be introduced at high enough concentration to effectively compete with oligo(dA) blocks for surface adsorption sites.

The physical properties of a DNA immobilization solution include mechanical (density, viscosity, surface tension, etc.), electronic (conductivity, dielectric constant, etc.), optical (index of refraction, transparency, turbidity, optical density, optical extinction coefficient, etc.), colligative (vapor pressure, freezing and boiling points, osmotic pressure), and magnetic (induced and remnant magnetization, permeability, susceptibility, etc.)

Whereas some of the physical properties will affect the ability to prepare, characterize, manipulate, and deliver the immobilization solution to place it in contact with a surface, the respective limits on those properties are determined by the specific techniques used to prepare, characterize, manipulate, and deliver the immobilization solution in each case.

For the purpose of this immobilization method, the only requirement placed on the physical properties of an immobilization solution is that it can be prepared, characterized, manipulated, and delivered to be placed in contact with the surface under the conditions specific to the chosen method.

Some of the physical properties will affect the requirements for the experimental parameters and conditions used for DNA immobilization: e.g., a high-viscosity immobilization solution may require higher temperature, longer deposition time, or mechanical agitation to produce the desired surface density of immobilized ssDNA.

Experimental parameters and conditions that can effect DNA immobilization include temperature, pressure, mechanical and/or convective agitation, static or dynamic exposure/delivery methods and immobilization (deposition) time. In general, effects of all the parameters and conditions on ssDNA immobilization are interdependent, and accordingly the combined effect of all the experimental parameters and conditions must be taken into account.

DNA immobilization has, in general, a non-linear dependence on the temperature of the immobilization solution. The lowest and highest physical limits of the immobilization solution temperature are given by its freezing and boiling points, respectively. The stability against thermal decomposition of the ssDNA used in a specific procedure provides another upper limit of the immobilization solution temperature. At low temperatures close to the freezing point of the immobilization solution, the delivery of ssDNA to the surface by bulk diffusion will be suppressed; therefore mechanical agitation and long deposition times may be required to achieve the desired surface density of immobilized ssDNA. At intermediate temperatures, approximately between 20° C. (typical “ambient” or “room” temperature) and 37° C. (human physiological temperature), increasing the temperature will, in general, enhance the delivery of ssDNA to the surface by bulk diffusion, therefore mechanical agitation may not be required and the desired surface density of immobilized ssDNA could be achieved using a shorter deposition time. For every combination of a DNA immobilization solution and a substrate material, there will be a critical solution temperature above which the DNA adsorption/desorption balance will shift towards desorption, thus decreasing the achievable surface density of immobilized ssDNA or even completely preventing DNA immobilization.

Pressure (either ambient or within a controlled-pressure vessel) will primarily affect the freezing and boiling points of a solution (thus increasing or decreasing the range of useable solution temperatures). Changing pressure will also potentially change one or more of the physical properties of a DNA immobilization solution.

Introducing or increasing the mechanical or convective agitation will, in comparision to a static solution, enhance the delivery of ssDNA to the surface, and thus can be used to compensate to some degree for a low solution temperature, low DNA concentration, low DNA diffusion constant, or short deposition time. The effect of introducing flow or other dynamic solution delivery/exposure methods is similar to that of agitation.

Increasing the immobilization time will in general, result in higher surface density of immobilized ssDNA. The specific deposition time used in any given procedure will be determined by other factors: e.g., the amount of time available the desired surface density of immobilized ssDNA, the substrate material, the presence of contaminants or additives competing for surface adsorption, etc. [Petrovykh et al., Quantitative Analysis and Characterization of DNA Immobilized on Gold, J. Am. Chem. Soc. 125, 5219-26 (2003)]

The objective of the DNA immobilization method described herein is to deterministically control the conformation and lateral spacing of the immobilized ssDNA. Accordingly, selecting the process parameters that are identified above as potentially having an effect of producing poorly-controlled DNA immobilization will, in general, limit the degree of deterministic control possible in a given system, or even completely eliminate the ability to control the immobilization. Conversely, selecting the process parameters that are identified above as potentially enhancing and speeding up the immobilization, or increasing the achievable surface density of immobilized ssDNA will, in general, lead to a higher degree of deterministic control over the conformation and lateral spacing of the immobilized ssDNA.

The method provides for the immobilization of oligonucleotides on a gold surface, whereby the attachment is provided by a block of n adenine nucleotides d(Aₙ) located at one end of an nucleotide sequence. A trivial sequence used as a lateral spacer in one embodiment of this method comprises only the said block of n adenine nucleotides d(Aₙ). In another embodiment, the rest of the sequence can be used as a “probe” (or “probe sequence”) in applications that make use of the ability of such a ssDNA probe to recognize and bind a complementary “target” sequence from solution. Under appropriate conditions, in addition to anchoring the nucleotides, the d(A) blocks in both embodiments of the method described above saturate nearly all available DNA-gold surface binding sites. As a result, this method produces surfaces that are inherently resistant to non-specific adsorption of other DNA, which includes the functional portions of the immobilized probes and any DNA in solution. This property is expected to decrease problems resulting from non-specific binding and also to enhance hybridization efficiency.

Immobilization via d(A) blocks provides multiple anchoring points to the gold surface for the immobilized nucleotides, thus the resulting attachment is more stable than traditional single-point attachment via a single thiol linker. The enhanced stability has been confirmed both against exposure to a buffer solution at an elevated temperature and against exposure to a solution of mercaptohexanol (MCH).

The d(A) block immobilization method does not require adding chemically-modified nucleobases/nucleotides or functional groups. It involves incorporating additional dA nucleotides into a sequence, which is significantly less expensive than most post-synthesis chemical modifications. No special reagents are required during synthesis, purification, or before or during the immobilization. In contrast, the traditional thiol modification is expensive, requires extra synthetic steps and a de-protection and purification step prior to use. A common contaminant introduced during preparation of thiol-modified DNA is dithiothreitol (DTT). This compound readily contaminates gold surfaces and can strongly suppress DNA immobilization on gold [Lee et al., Evidence of Impurities in Thiolated Single-Stranded DNA Oligomers and Their Effect on DNA Self-Assembly on Gold, Langmuir 21, 5134-41 (2005)].

Immobilization via d(A) blocks and its variant allows preparation of DNA surface probe densities below 10¹³ cm⁻², while ensuring that the probe sequences project into the solution. Both these factors are known to increase the efficiency of hybridization with complementary targets from solution. Furthermore, the combination of both the grafting density below 10¹³ cm⁻² and the brush-like conformation of the probe sequences provided by this method is particularly beneficial for DNA hybridization applications, but is difficult to reliably produce by other existing methods.

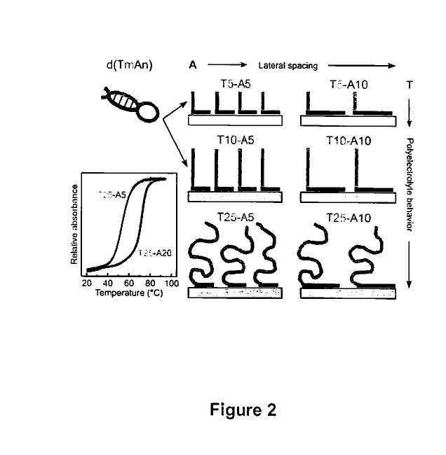

In order to elucidate the mechanism underlying the methods described above, the adsorption of model block oligonucleotides, d(Tₘ-Aₙ), with systematically varied thymine (dT) and adenine d(A) nucleotide block lengths m and n, on gold surfaces was studied. In this model system, the d(T) blocks act as trivial probe sequences, the grafting density and conformation of which can be independently verified and quantified using X-ray photoelectron spectroscopy (XPS) and Fourier transform infrared spectroscopy (FTIR). These quantitative measurements (described in detail in Example 1) confirmed an adsorption model (L-shape model) where the d(A) blocks preferentially adsorb on the gold substrate and the d(T) blocks extend away from the substrate. The grafting density of short oligos, such as d(T₅-A₅), d(T₁₀-A₅). d(T₅-A₁₀), d(T₁₀-A₁₀), is specifically and linearly determined by the length of the d(A) block (FIG. 2). In all cases, the block-oligos with longer d(A) blocks exhibit lower grafting densities.

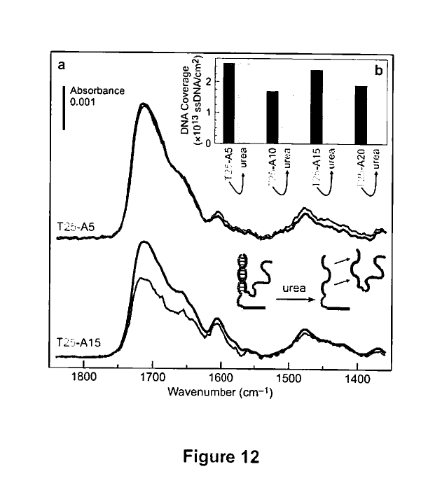

Films obtained using oligos with long d(T) blocks, such as d(T₂₅-A₅) and d(T₂₅-A₁₀) display the same trend of decreasing grafting density with increasing length of the d(A) block, but overall have lower grafting densities than short block oligos—an effect attributed to steric and electrostatic repulsion between the longer d(T₂₅) brush strands, which can no longer be approximated as an extended chains, but instead behave as anchored random coils (FIG. 2). Because of the complementary nature of the d(Tₘ-Aₙ) oligos, films of oligos with both long d(T) and long d(A) blocks, such as d(T₂₅-A₁₅) and d(T₂₅-A₂₀), contain stable hybrids. Exposing these films to denaturing conditions recovers the d(T₂₅) brush structure, i.e., the structure with d(T) blocks extending away from the surface, as shown in FIG. 12.

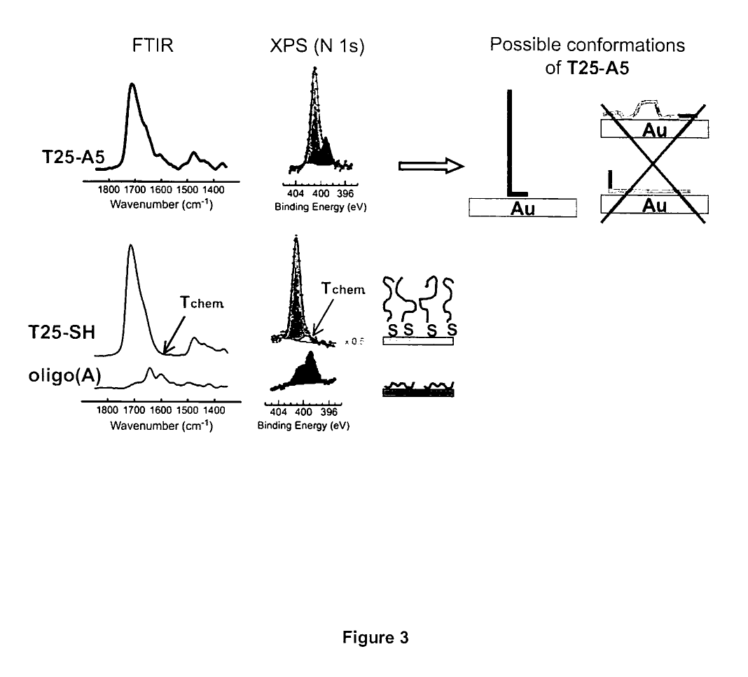

The model experiments with d(Tₘ-Aₙ) block-oligos demonstrate that dA nucleotides can be used to anchor DNA probes on gold surfaces, and to generate stable brush-like layers with controlled structure, without the use of a thiol linker. The brush-like conformation of block-oligos is explained by the fact that thymine oligos (dT) adsorb much more weakly to gold than do adenine (dA) oligos. The large difference in adsorption affinity between dA and dT results in saturation block-oligo surface densities that are strongly dependent on the dA/dT content of the d(Tₘ-Aₙ) block-oligos, both unmodified and thiol-modified (—SH). The limiting cases of (dT)ₘ—SH and (dA)ₙ—SH thiol-modified homo-oligos tend to “stand upright” on gold as anchored random coils with high grafting density and to lie flat on the surface, respectively (FIG. 3).

In general, for a polyelectrolyte chain molecule, such as ssDNA, adsorbed on a surface, upright “brush-like” conformations are not expected until the grafting density is high enough that repulsive interactions between overlapping chain segments force the chains to stretch away from the surface. In other words, in general, for polyelectrolyte chains adsorbed on a surface, the grafting density and conformation cannot be independently controlled. The typical immobilization of thiol-modified ssDNA falls into this category of systems, for example. Immobilization via d(A) blocks introduces the critical ability to decouple the control of these two characteristics. Namely, the grafting density is controlled by the length of the d(A) attachment block. In turn, the brush-like conformation of the probe sequence is provided because the adsorbed d(A) blocks saturate the DNA-Au surface binding sites—the characteristic inherent to this immobilization method and therefore one independent of the grafting density. In more general terms, the independent control of the grafting density and conformation provides a possibility for controlling the (repulsive) nearest-neighbor interactions between anionic sugar-phosphate backbones in ssDNA films within a range of values between the strong interactions typical for a close-packed monolayer of thiol-modified oligo(dT), for example, and the all but negligible nearest-neighbor repulsion of low grafting density ssDNA brushes.

The high intrinsic affinity of oligo(dA) for gold and the resulting tendency of oligo(dA) to dominate the surface adsorption in various competitive adsorption/displacement environments leads to three types of applications.

First, the method provided herein provides for attachment of ssDNA probes with controlled immobilization density and conformation. A block of n dA nucleotides [d(Aₙ)] is incorporated at either the 5′ or 3′ end of a synthetic oligonucleotide. Upon exposure of a gold surface under appropriate conditions to an aqueous buffer solution of such a nucleotide, the d(Aₙ) block attaches to the surface and the remaining part of the sequence extends into the solution, adopting an approximately L-shape conformation (FIG. 2).

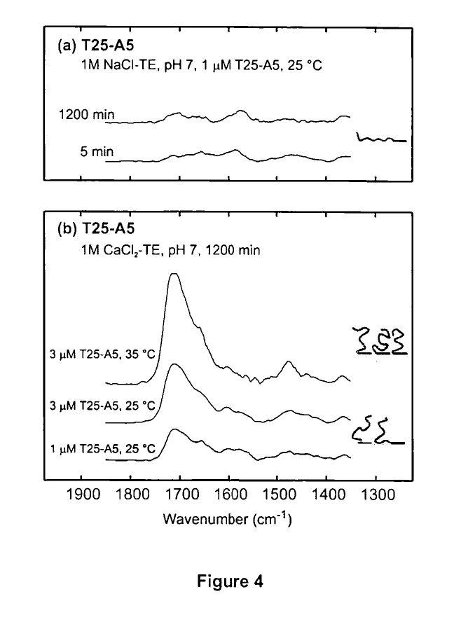

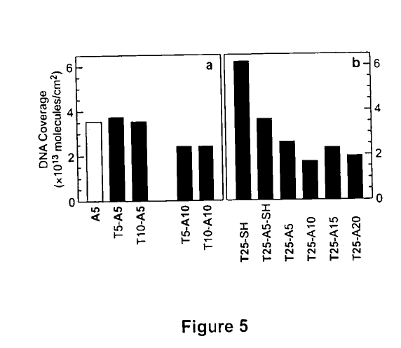

Two primary driving forces lead to the L-shape conformation shown in FIG. 2. First, once a sufficiently high fraction of the gold surface is covered by the chemisorbed d(A) blocks, the d(T) blocks are prevented from binding to the surface, and thus are forced to project into the solution. Second, under appropriate deposition conditions, d(T) blocks extending into the solution are further stabilized, since such configuration lowers DNA-DNA electrostatic repulsion. As FIG. 4 demonstrates, a combination of relatively high DNA concentration (3 μM) and high concentration of a divalent buffer salt (1 M CaCl₂) at elevated temperature was used to force the immobilized probes to adopt the L-shape conformation. Under these conditions, both factors leading to an L-shape conformation are enhanced. However, once the immobilization is completed, the probes retain the L-shape conformation, even if the buffer conditions change. The grafting density of the immobilized block-oligos is controlled primarily via the length of d(A) blocks, as shown in FIG. 5.

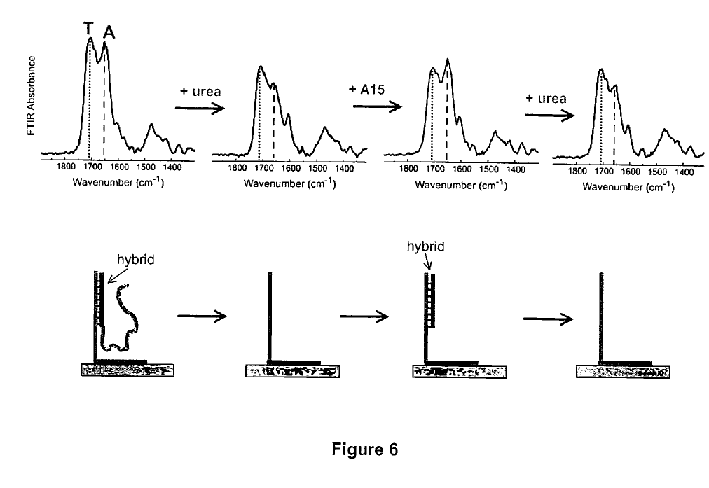

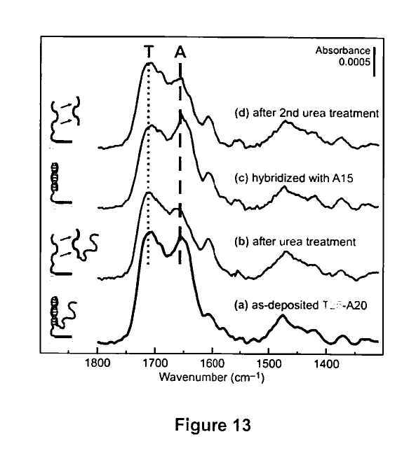

The resulting ssDNA probes can be used for hybridization as demonstrated in a series of experiments as shown in FIG. 6 and FIG. 13. By following the evolution of spectral features corresponding to T and A bases in FTIR spectra (green and red dashed lines, respectively) the reversible hybridization sequence schematically depicted at the bottom of FIG. 6 can be established. As-deposited films of d(T₂₅-A₂₀) oligos contain some co-adsorbed hybrids. These hybrids can be denatured by exposure to an 8 M urea solution at room temperature. After the urea treatment the unattached probes are removed by a deionized water rinse. The remaining probes are still in the L-shape conformation and available for hybridization, as confirmed by the spectral change upon exposure to a solution of a complementary (dA)₁₅ oligo. In this case, only the peak corresponding to A bases increases. Finally, the second hybridization step is shown to be reversible by denaturing the resulting hybrids using a second urea treatment.

The second type of application provided by the methods described herein provides for controlling the density of ssDNA probes by coadsorption with and/or displacement by oligo(dA). Increasing the length of the d(A) block allows a controlled decrease in grafting density of DNA probes down to approximately 10¹³ cm⁻², as shown in FIG. 5. A grafting density of approximately 1-5×10¹² cm⁻² is considered optimal for hybridization. Achieving such low densities via increasing the length of d(A) blocks alone would require impractically long d(A) blocks. A simple modification of the procedure described in the next paragraph yields a practical alternative for controlling the surface density below 10¹³ cm⁻², while maintaining the L-shape conformation of the probes.

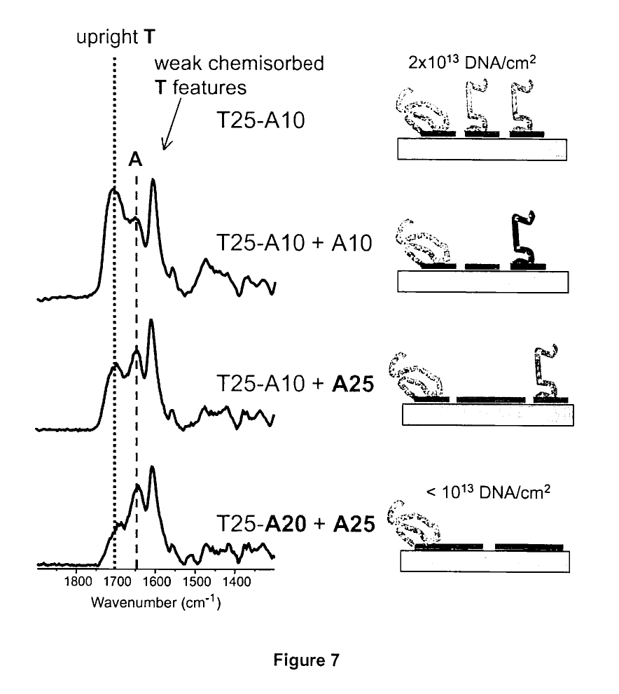

This method calls for adding oligo(dA) to the immobilization solution of DNA functionalized with d(A) blocks. The practical implementation is illustrated for mixtures of d(T₂₅-Aₙ) probe oligos with (dA)ₖ, where n and k represent the number of dA nucleotides in the anchoring d(A) blocks and in the oligo(da) lateral spacers, respectively, as shown in FIG. 7. Because oligo(dA) effectively competes with [the anchoring d(A) blocks of] d(Tₘ-Aₙ) for adsorption, the d(T₂₅-Aₙ) DNA probes are essentially diluted by oligo(dA) on the surface. Note that the total amount of dA nucleotides is nearly constant in all cases, confirming that the resulting surface is still saturated with dA nucleotides, which forces the d(T₂₅-Aₙ) probes into the L-shape conformation, even at these low grafting densities of the d(T) probe sequences, which can be readily estimated from the absorbance value of the nonchemisorbed dT feature. The grafting density of the probe sequences can be controlled in several ways when using this method. First, the concentration of the lateral spacer oligo(s) relative to the probe oligo(s) can be increased or decreased to, respectively, decrease or increase the grafting density of the probe sequences. Second, the length of the spacer oligo(s) relative to that of the anchoring oligo(s) can be adjusted. Third, the duration of the coadsorption/displacement treatment and the time that it is started, relative to the beginning of the immobilization, can too be adjusted. Finally any combination of these control methods can be used. The availability of multiple control parameters opens the possibility of multiplexing the effects, i.e., by adding two probe oligos with different lengths of the anchoring d(A) blocks and allowing them to adsorb competitively against each other and against the lateral spacer oligo.

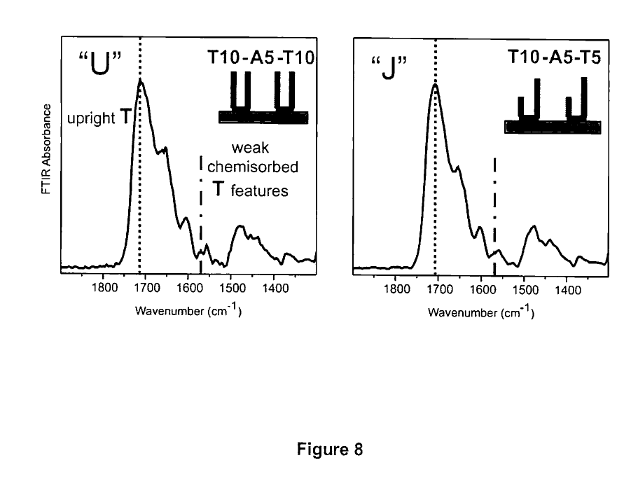

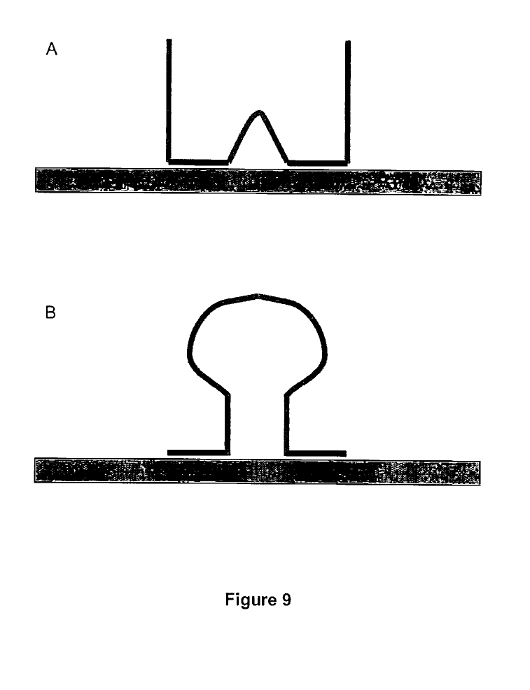

The third type of application provided by the methods described herein provides for a method of immobilizing DNA in complex conformations. Synthetic ssDNA can be produced or purchased with one or more d(A) blocks positioned between other sequences, rather than simply at either 5′ or 3′ ends. Building on the L-shape analogy, if a d(A) block is placed in the middle of an oligonucleotide attachment via that d(A) block results in a “U-shape” conformation, with both 5′ and 3′ ends of the oligonucleotide projecting into the solution. FIG. 8 shows an example for a d(T₁₀-A₅-T₁₀) oligo. Similarly, if the 5′ and 3′ end sequences are of unequal length, the d(A) block attachment results in a “J-shape” conformation (example for a d(T₁₀-A₅-T₅) oligo shown in FIG. 8). Introducing two separate d(A) blocks into the sequence will result in a “W-shape” or “Ω-shape” conformation, depending on whether the two d(A) blocks are in the middle of the sequence or at the ends, as shown in FIGS. 9a and 9b respectively. Those skilled in the art would understand that by placing a specific number of d(A) blocks of specific length(s) at specific locations in a sequence, a variety of complicated and robust DNA conformations can be thus reliably achieved on gold surfaces. Those skilled in the art would also recognize that by this method controlled mixtures of two or more probe sequences can be immobilized on a surface with perfect mixing ratios. Because the mixing ratios in this method are deterministic rather then statistical, e.g., the two functional sequences immobilized in a U-shape conformation are guaranteed to be within a fixed distance from one another, the perfect mixing ratios can be achieved at any overall grafting density, from the most dilute single-molecule limit to close-packed brushes at saturation density.

Having described the invention, the following examples are given as particular embodiments thereof and to demonstrate the practice and advantages thereof. It is understood that the examples are given by way of illustration and are not intended to limit the specification or the claims in any manner.

While the exact optimal ranges of process parameter values will be determined by the specific requirements and the desired surface density and conformation of immobilized ssDNA, the following process parameters have been found both practical and useful. The ssDNA concentration ranges from about 1 to about 3 μM. The length n of the anchoring (dAₙ) block ranges from about 5 to about 25. The length l of the lateral-spacer (dAₙ) block ranges from about 5 to about 25. The length m of the functional sequence ranges from about 5 to about 35. The immobilization solution volume ranges from about 1 to about 5 mL for a 1 cm² sample. The immobilization solution temperature ranges from about 20° C. to about 35° C. The salt added to immobilization solution can be NaCl, KCl. K₂HPO₄, KH₂PO₄, CaCl₂, or MgCl₂. The salt concentration ranges from about 1 to about 3 M. The buffering agent was Tris-HCl. 1-10 mM. The buffered solution pH was about neutral. The chelator was EDTA, at concentrations ranging from about 1 to about 10 mM. The deposition time ranged from about 1 to about 40 hours.

The highest degree of deterministic control was achieved in model systems with the following parameters. The aqueous solution used was deionized water (18.3 MΩ), with a ssDNA concentration of about 3 μM. The length n of the anchoring (dAₙ) block ranged from 5 to 20. The length l of the lateral-spacer (dAₗ) block ranged from about 5 to about 25. The length m of the functional sequence ranged from about 5 to about 25. The immobilization solution volume was 2 mL for a 1 cm² sample. The immobilization solution temperature was about 35° C. The salt added to immobilization solution was 1M CaCl₂. The buffering agent used was Tris-HCl, 10 mM. The buffered solution had a neutral pH. The chelator used was EDTA, 1 mM. The deposition time was about 40 hours. The substrate used was a polycrystalline Au film sputter-deposited on Si(100) wafers. The substrate was cleaned using a “piranha” solution [70% H₂SO₄ 30% H₂O₂ (30% H₂O₂ in H₂O)]

### EXAMPLE 1

Commercial custom oligonucleotides were synthesized and HPLC purified by the vendor and used as-received without further purification. The d(Tₘ-Aₙ) oligos are written in the 3′ to 5′ direction where m and n are the number of nucleotides in the dT and dA blocks, respectively. For ease of presentation, these oligos are written as “Tm-An” in the Figures. The 5′ alkanethiol modified oligonucleotides [(dT)₂₅—SH, (dA)₅—SH, (dA)₂₅—SH, and d(T₂₅-A₅)—SH] were used without removing the protective S—(CH₂)₆0H group from the 5′ end. Buffer solutions were prepared containing 1×TE (10 mM Tris-HCl, 1 mM EDTA), 1 M NaCl or 1 M CaCl₂, and were adjusted to pH 7 with HCl. An aqueous 8 M urea solution and an aqueous 1 mM 6-mercapto-1-hexanol (MCH) solution were used for post-deposition treatments.

Preparation of DNA-coated gold films. Polycrystalline gold films on single-crystal Si(100) wafers were used as substrates. Prior to the deposition of gold, the wafers were cleaned using a “piranha solution” consisting of 70% H₂SO₄ and 30% H₂O. (30% H₂O₂ in H₂O). After cleaning, a 20 nm Cr adhesion layer was deposited by vapor deposition, followed by 200 nm of Au. Each substrate was again cleaned with piranha solution and rinsed thoroughly with deionized water (18.3 MΩ) immediately prior to adsorption of DNA. For the d(Tₘ-Aₙ) films, clean gold substrates (approximately 1 cm² each) were immersed in 3 μM DNA in 1 M CaCl₂, pH 7, buffer solutions at 35° C. (solution volume 2 mL). These conditions were found to produce films with near-saturation surface densities of d(A) blocks. Before analysis, each sample was rinsed with deionized water to remove buffer salt and loosely bound DNA, and blown dry under flowing nitrogen. A set of samples was also immersed in 8 M urea solution to test for the presence of adsorbed DNA hybrids.

FTIR measurements. Infrared reflection absorption spectra were obtained using an FTIR spectrometer equipped with a wire grid infrared polarizer (p-polarized) and a variable angle specular reflectance accessory (75° grazing incidence angle) as described in Petrovykh et al., Quantitative Analysis and Characterization of DNA Immobilized on Gold, J. Am. Chem. Soc. 125, 5219-26 (2003). FTIR measurements were performed in a nitrogen purged environment using freshly prepared samples and a piranha cleaned gold substrate as a reference.

XPS measurements were performed using a commercial XPS system equipped with a monochromatic Al Kα source, a hemispherical electron energy analyzer (58° angle between monochromator and analyzer), and a magnetic electron lens. A detailed descriptions of the quantitative XPS analysis of DNA adsorbed on gold is available in Petrovykh et al., Quantitative Analysis and Characterization of DNA Immobilized on Gold. J. Am. Chem. Soc. 125, 5219-26 (2003), and Petrovykh et al., Quantitative Characterization of DNA Films by XPS, Langmuir 20, 429-40 (2004), both incorporated herein in full by reference.

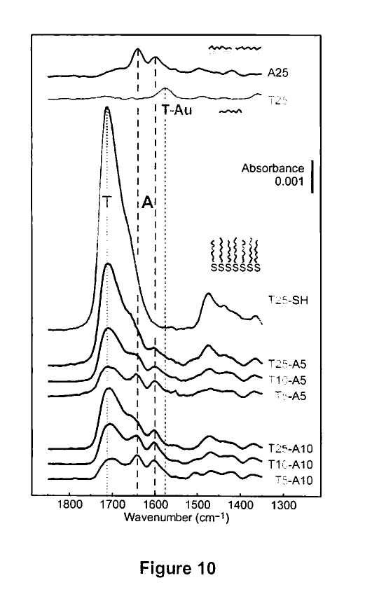

Reflectance FTIR spectra are shown in FIG. 10 for a series of d(Tₘ-Aₙ) oligos adsorbed on gold substrates. The 1300 cm⁻¹ to 1900 cm⁻¹ spectral region contains vibrational features that are primarily associated with the DNA bases. The features used to interpret the FTIR data are identified in the reference spectra obtained from (dT)₂₅ and (dA)₂₅ homo-oligos, and alkanethiol modified (dT)₂₅—SH adsorbed on gold (top three spectra in FIG. 10). The two features at approximately 1600 cm⁻¹ and approximately 1650 cm⁻¹ in the (dA)₂₅ spectrum are characteristic of dA adsorbed on gold. The primary features in the (dT)₂₅ spectrum are located between 1550 cm⁻¹ and 1600 cm⁻¹, and have been attributed in the literature to carbonyl groups in the thymine bases that interact with the gold substrate (hereafter referred to as the chemisorbed dT feature). The dominant feature at approximately 1700 cm⁻¹ in the (dT)₂₅—SH spectrum has been attributed to the C═O vibration from carbonyl groups in the thymine base that are not directly adsorbed on the gold surface (hereafter referred to as the nonchemisorbed dT feature).

For the d(Tₘ-Aₙ) samples, three trends in the FTIR spectra are observed. First the absorbance values of the features associated with dA (red dotted lines) are similar for the six samples and are very similar to those measured for the (dA)₂₅ homo-oligo. Second, for a series of samples with fixed d(A) length (e.g. T₅-A₅, T₁₀-A₅, T₂₅-A₅), the nonchemisorbed dT feature (at approximately 1700 cm⁻¹) increases as the length of the d(T) block increases. The third trend is that fixing the length of the d(T) block and increasing the length of the d(A) block (e.g. T₁₀-A₅ and T₁₀-A₁₀) leads to a decrease in the absorbance of the nonchemisorbed dT feature (at approximately 1700 cm⁻¹). Note that the absence of the chemisorbed dT feature in the (dT)₂₅—SH spectrum indicates that (dT)₂₅—SH oligos anchor on gold via the thiol group and that few dT nucleotides directly adsorb on the gold. In the d(Tₘ-Aₙ) spectra, although the chemisorbed dT features overlap somewhat with dA features, the absorbance in that frequency region is generally small and is similar to what is observed for the (dA)₂₅ spectrum.

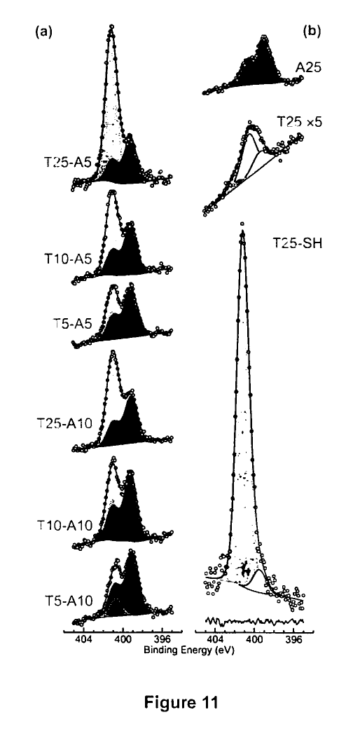

XPS was used to obtain quantitative information about the stoichiometry and molecular coverage of the DNA films. Spectra of the N 1s region are presented in FIG. 11a for the d(Tₘ-A₅) and d(Tₘ-A₁₀) oligo series analogous to those examined by FTIR. For reference, spectra from unmodified (dA)₂₅ and (dT)₂₅, and thiol-modified (dT)₂₅—SH oligos are shown in FIG. 11b. The two main features in these spectra are emphasized by shading. The first is a single peak with BE approximately 401 eV (light shading). The reference spectrum from the (dT)₂₅—SH film indicates that this peak corresponds to dT not directly adsorbed on the gold surface (nonchemisorbed dT). The second component is a pair of peaks (dark shading, BE approximately 399 eV), which is a typical N 1s spectral envelope for dA, as shown by the reference (dA)₂₅ spectrum in FIG. 11b. This N 1s spectral signature of dA is invariant with respect to the adsorption conditions and to the film structure, and thus the dA spectral envelope can be fixed in fitting, allowing for simplified deconvolution of N 1s spectra for d(Tₘ-Aₙ) oligos (FIG. 11a). The reference spectra for (dT)₂₅ and (dT)₂₅—SH oligos also contain lower BE components associated with dT chem sorbed on gold (light shading with black outline). In agreement with the FTIR results, only a small fraction of the bases in the (dT)₂₅—SH film are chemisorbed, whereas in the unmodified (dT)₂₅ film the bases are almost fully chemisorbed, indicating that the (dT)₂₅ oligos essentially lie flat on the gold surface.

All XPS spectra are shown normalized by the respective Au 4f_{7/2} substrate peak intensities, so that the corresponding N 1s peak areas are approximately proportional to nucleotide coverages. Because adenine contains five nitrogen atoms and thymine—only two, for an equal number of bases the area of the dA (red) component is approximately 2.5 times that of the dT, which can be clearly seen in d(T₅-A₅) and d(T₁₀-A₁₀) fits. The (dT)₂₅ nucleotide coverage is much smaller than for (dA)₂₅ (the (dT)₂₅ [N 1s spectrum is scaled up by a factor of 5, as indicated in FIG. 11b)], consistent with the low dT-Au affinity, as previously discussed.

DNA film thicknesses were calculated based on the attenuation of absolute intensities of the substrate Au 4f and Au 4d peaks. The nitrogen atomic density in each DNA film was then calculated relative to the atomic density of the gold substrate from the simple uniform overlayer model, relative peak intensities and film thicknesses. Finally, the DNA coverage (grafting density) was calculated from the nitrogen atomic density and film thickness, assuming ideal molecular stoichiometry for DNA. The detailed description of the quantitative analysis of DNA films by XPS is given in Petrovykh et al., Quantitative Characterization of DNA Films by XPS, Langmuir 20, 429-40 (2004).

The FTIR and XPS results provide complementary evidence for the formation of d(Tₘ-Aₙ) brushes where the d(A) blocks adsorb on the gold surface and the d(T) blocks extend away from the surface, as illustrated by the diagrams in FIG. 2, which are idealized representations of the actual oligo conformation. This basic conformation can be deduced from the dT features in the FTIR and XPS spectra shown in FIGS. 3, 10 and 11. In each of the d(Tₘ-Aₙ) spectra, the presence of the nonchemisorbed dT feature (1700 cm⁻¹ in FTIR and 401 eV in XPS) and the absence of the chemisorbed dT feature (1550 cm⁻¹ to 1600 cm⁻¹ in FTIR and 398 eV to 399 eV in XPS) indicate that few thymine bases interact with the gold surface. The deduced conformation of these block-oligos is one, in which the d(T) blocks extend away from the surface and are anchored to the substrate by the d(A) blocks.

That d(Tₘ-Aₙ) oligos adsorb in this fashion might be considered surprising, because adenine and thymine are complementary nucleobases. In solution, the d(Tₘ-Aₙ) oligos can self-interact with other d(Tₘ-Aₙ) oligos to form hairpin and multistrand structures, as shown in FIG. 2. One might expect the presence of these structures to impede the formation of the L-shaped conformation. However, previously reported competitive adsorption experiments between (dA) and (dT) homo-oligos have shown that (dA) oligos preferentially adsorb on gold surfaces, even in the presence of a 10-fold excess of (dT) complementary oligos. Experiments described in Kimura-Suda et al., Base-Dependent Competitive Adsorption of Single-Stranded DNA on Gold. J. Am. Chem. Soc. 125, 9014-5 (2003) demonstrated that the gold surface induces the denaturing of (dA)·(dT) hybrids and suggests that intra- or inter- strand interactions between d(Tₘ-Aₙ) oligos in solution should not prevent the adsorption of d(Tₘ-Aₙ) oligos on gold via blocks of dA nucleotides.

For surfaces with lower DNA coverages, prepared using either shorter immobilization times or lower ionic strength buffers, spectral features associated with chemisorbed thymine are observed, as shown in FIG. 4. The presence of the chemisorbed dT features indicates that although the d(Tₘ-Aₙ) oligos ultimately adapt an L-shaped conformation, they initially adsorb more or less flat on the surface, similar to what was previously reported for (dT)₂₅—SH oligos [Petrovykh et al., Quantitative Analysis and Characterization of DNA Immobilized on Gold, J. Am. Chem. Soc. 125, 5219-26 (2003)].

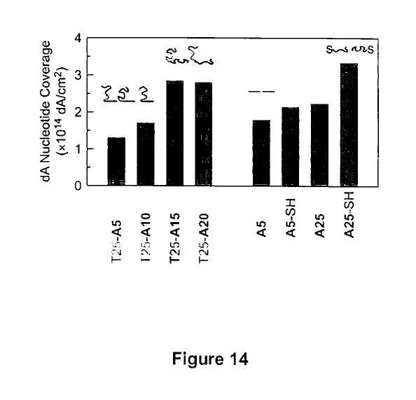

FIG. 5a and FIG. 14 demonstrates that the dA nucleotide coverage on gold is largely independent of the d(A) block length. If that assumption holds, then the grafting density of d(T) brush strands is controlled by the length of the d(A) block. The high dA-Au affinity forces the d(A) blocks to adsorb nearly flat on the gold surface and occupy virtually every available dA-Au binding site. Coverage measurements by XPS for (dA) homo-oligos support this assumption. A 400% length increase between (dA)₅ and (dA)₂₅ results in a dA nucleotide coverage increase of only 20% (1.8×10¹³ cm⁻² vs. 2.2×10¹³ cm⁻²) as shown in FIG. 14. In addition, dA nucleotide coverages depend only weakly on the immobilization conditions, including the choice of buffer. Buffer-dependent variations in charge screening are expected to have little effect on the quantity of adsorbed strands that lie flat on the gold surface, while having a much larger effect on grafting densities for a film of DNA strands projecting into the solution.

The simple model in FIG. 2 predicts that DNA grafting density should remain the same for oligos with the same d(A) block length, and should decrease as the d(A) length increases. The data for the short d(Tₘ-Aₙ) strands quantitatively follow this model (T₅-A₅, T₁₀-A₅, T₅-A₁₀, and T₁₀-A₁₀, as shown in FIG. 5a). Comparing the d(T₅-A₅) and d(T₁₀-A₅) samples, the grafting density is nearly identical to the DNA coverage measured for (dA)₅. The coverage decreases by approximately 35% when the d(A) length is increased to 10 nucleotides for the d(T₅-A₁₀) and d(T₁₀-A₁₀) oligos which have identical grafting densities.

The FTIR spectra of FIG. 10 support this interpretation. For fixed d(A) length, increasing the d(T) length from 5 to 10 nucleotides results in a factor of two increase in the intensity of the nonchemisorbed dT feature, exactly as expected if the grafting density is the same in these two films. For fixed d(T) length, increasing the d(A) length from 5 to 10 nucleotides leads to a decrease in the absorbance of the dT feature in the FTIR spectra—indicating fewer dT nucleotides and a lower grafting density. Thus short d(Tₘ-Aₙ) oligos follow the model presented in FIG. 2, and that the primary factor controlling the grafting density of these oligos is the length of the d(A) block.

FIG. 5b shows the effect of increasing the length of the linker, X, where X═—SH or d(A)ₙ, on the grafting density of relatively long d(T)₂₅ brush strands. Grafting density should decrease as the length of the d(A) block increases. The two thiol modified oligos, d(T)₂₅—SH and the thiol modified d(T₂₅-A₅) film, d(T₂₅-A₅)—SH, have the highest grafting densities. These are followed by the d(T₂₅-A₅) film and the d(T₂₅-A₁₀) film, as predicted.

Quantitatively, the grafting density measured for d(T₂₅-A₅) should be similar to that measured for the shorter d(T₅-A₅) and d(T₁₀-A₅) oligos, that is grafting density of d(Tₘ-A₅) oligos should be independent of the length of the d(Tₘ) block. However the grafting density of adsorbed d(T₂₅-A₅) oligos is approximately 30% lower than that measured for the d(T₁₀-A₅) and d(T₅-A₅) films, as shown in FIG. 5. A similar trend is observed comparing the d(T₂₅-A₁₀) film to the d(T₁₀-A₁₀) and d(T₅-A₁₀) films, as shown in FIG. 5. The decrease in grafting density with increasing length of d(T) block is attributed to the electrostatic and entropic forces that must be overcome to uncoil and pack the longer d(T) brush strands. This trend, a decrease in grafting density with increase in length, has been reported for thiol modified ssDNA and attributed in part to the larger radius of gyration of longer brush strands [Petrovykh et al., Nucleobase Orientation and Ordering in Films of Single-Stranded DNA on Gold, J. Am. Chem. Soc. 128, 2-3 (2006)]. In other words, for d(T₂₅-A₅) and d(T₂₅-A₁₀) films the grafting density is not solely determined by the length of the dA block, but also by polyelectrolyte behavior of the d(T) brush strands.

The oligos with the longest d(Aₙ) blocks, d(T₂₅-A₁₅) and d(T₂₅-A₂₀), are predicted to have the lowest grafting densities of all the d(Tₘ-Aₙ) studied. However, “as-deposited” coverages measured for these two films are actually as high as those measured for d(T₂₅-A₅) and d(T₂₅-A₁₀) films, respectively, as shown in FIG. 5b. Due to the complementary nature of d(Tₘ-Aₙ) oligos, hybrids can exist between between d(T) blocks in the adsorbed d(Tₘ-Aₙ) oligos and d(A) blocks of oligos as shown in FIG. 12a inset. Ex situ FTIR and XPS can only detect such structures if they are not disrupted by the post-deposition stringency rinse; thus the ability to observe hybrids depends on their stability in deionized water, which in this case is determined by the lengths of the dA and dT blocks. Hybrids of d(Tₘ-Aₙ) oligos with dT and dA blocks of ten or fewer nucleotides have melting temperatures near or below room temperature, and are likely to be denatured and removed by the deionized water rinse. The d(T)₂₅ blocks of d(T₂₅-A₁₅) and d(T₂₅-A₂₀) adsorbed oligos can interact with oligos from solution to form longer hybrids with overlaps up to 15 or 20 bases, respectively. These more stable hybrids appear to withstand the deionized water rinse, and, thus, are observed by FTIR and XPS.

To test for hybridized structures, a set of d(T₂₅-Aₙ) samples was soaked in an 8 M urea denaturing solution for 30 minutes. FTIR spectra for the d(T₂₅-A₅), d(T₂₅-A₁₅) and d(T₂₅-A₂₀) films on gold “as-deposited” and after soaking in urea are shown in FIG. 12a. The d(T₂₅-A₅) spectra are identical for “as-deposited” and urea-treated samples, indicating that essentially no hybrids are present after the deionized water rinse, as expected. Similar results were obtained for d(T₂₅-A₁₀). XPS coverage data obtained from both of those samples confirms that no DNA was removed by the urea treatment.

By contrast, after the urea treatment, the d(T₂₅-A₁₅) and d(T₂₅-A₂₀) samples show reduction in absorbance for the peaks associated with dA and non-chemisorbed dT. Oligo coverages for these samples decrease after the urea treatment by approximately 20% and approximately 25% for the d(T₂₅-A₁₅) and d(T₂₅-A₂₀) samples, respectively. Control experiments show that (dA)₁₅ oligos can be hybridized to the d(T₂₅-Aₙ) films and are removed by subsequently soaking the film in urea (FIGS. 6 and 13). Therefore, “as deposited” d(T₂₅-A₁₅) and d(T₂₅-A₂₀) samples contain excess oligos coadsorbed via hybridization. After removal of these hybrids by urea, the data in FIG. 12b show that the d(T₂₅-A₂₀) oligo has the lowest grafting density, as predicted by the model. Importantly, we observe that the spectrum of d(T₂₅-A₂₀) contains no evidence of chemisorbed dT. This observation contrasts the behavior of (dT)₂₅—SH, where at comparable (dT) grafting densities a significant fraction of the dT nucleotides are chemisorbed to the substrate. Thus, dT brush strands anchored by dA nucleotides can exist in an upright orientation at lower grafting densities in comparision to dT strands attached by thiol linkers. Presumably for d(Tₘ-Aₙ) adsorption on gold via d(A) block attachment, at saturation coverages the dA nucleotides occupy all the DNA-gold surface binding sites, thereby preventing nonspecific interactions between dT nucleotides and the gold surface.

The grafting density of d(T₂₅-A₅)—SH is higher than that of the d(T₂₅-A₅) film (see FIG. 5b). Higher coverages are also measured for thiol-modified (dA)₅—SH and (dA)₂₅—SH films compared to the corresponding unmodified (dA)₅ and (dA)₂₅ films, as shown in FIG. 14, suggesting that the thiol linkage has a slightly higher affinity for gold compared to dA. FIG. 14 also presents the dA nucleotide coverage in the d(T₂₅-A,) films. Although the oligo grafting density tends to decrease as the length of the dA block increases (FIG. 5), the dA nucleotide coverage increases slightly as the number of nucleotides in the d(A) block increases. The trends in coverage variation with length and thiol-modification for the d(T₂₅-A,:) films are essentially the same as that observed for the (dA) homo-oligos shown in FIG. 14. This suggests that an additional degree of control can be provided by functionalizing the anchoring d(A) block with a moiety, which has high and/or specific affinity for the chosen surface. This modification may be particularly advantageous if the affinity for the surface of said moiety is higher than that of the d(A) block. All of the essential control aspects imparted by the use of the d(A) block will be retained, but the added moiety will provide for a higher initial sticking probability to the substrate and, therefore, for faster immobilization kinetics.

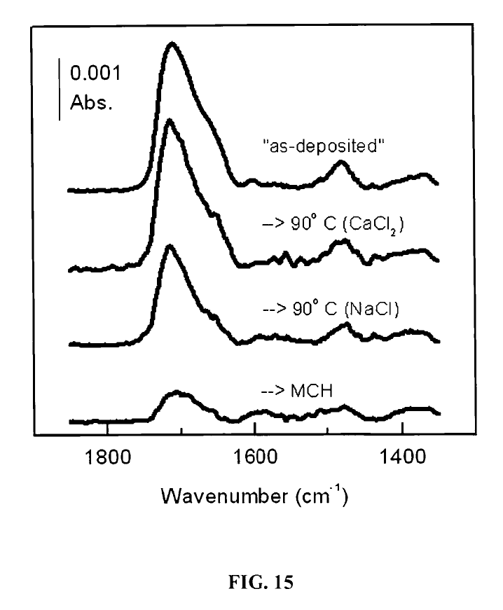

The stability of d(T₂₅-A₅) adsorbed on gold, which would be important to any practical application using d(A) blocks to immobilize ssDNA on gold, was assessed in two ways: (1) by exposing the d(T₂₅-A₅) films to solutions containing 6-mercapto-1-hexanol (MCH), and (2) by exposing the d(T₂₅-A₅) films to 90° C. buffer solutions for 30 min. FIG. 15 shows FTIR spectra obtained from a d(T₂₅-A₅) film after each of these treatments. For both situations, the major change seen in the FTIR spectra is an overall reduction of the nonchemisorbed dT feature. Experiments with (dA) and (dT) homo-oligos adsorbed on gold have shown that MCH effectively displaces adsorbed dA and dT nucleotides and can remove a substantial amount of thiol-bound DNA. The adsorbed d(T₂₅-A₅) oligos behave in a manner consistent with that observation. That is, after exposing the d(T₂₅-A₅) film to a 1 mM MCH/H₂O solution for 1 min, the nonchemisorbed dT peak decreased by approximately 80%, indicating most of the DNA film was displaced by the MCH. Similar results were observed for (dT)₂₅—SH films.

The temperature stability of the adsorbed d(T₂₅-A₅) is observed to be dependent on the identity of the counterion present in the solution. Little or no DNA was lost from the surface after soaking for 30 min in 1M CaCl₂-TE buffer at 90° C. However, after soaking for 30 min in 1 M NaCl-TE buffer at 90° C., the nonchemisorbed dT peak in the FTIR spectra decreased by approximately 30%, suggesting that some of the d(T₂₅-A₅) strands were removed from the surface. In a control experiment, soaking the films in room temperature 1 M NaCl-TE, pH 7 buffer solution resulted in no measured change in DNA coverage. In analogous experiments, the nonchemisorbed dT feature in FTIR spectra obtained from (dT)₂₅—SH films decreased by approximately 15% after soaking for 30 min in 1M CaCl₂-TE buffer at 90° C. and decreased by over 60% after soaking for 30 min in 1M NaCl-TE buffer at 90° C., which are significantly larger losses than those observed for d(T₂₅-A₅).

Thus, dT brushes attached to gold via d(A) blocks are at least as stable as those that are attached to gold by thiol linkers, particularly in terms of stability in solutions at elevated temperature. A likely mechanism for the enhanced stability of both DNA films in the 1 M CaCl₂-TE buffer solution, relative to the 1 M NaCl-TE buffer solution, is electrostatic cross-linking between dT portions of the adsorbed oligos induced by the divalent cations. The crosslinking would effectively give each strand multiple attachments to the gold through interstrand interactions which would have to be simultaneously broken in order to remove the strand from the surface. The greater stability of d(T₂₅-A₅) compared to (dT)₂₅—SH may be a result of multipoint dA attachments to the gold within each strand. This interpretation is consistent with the observation that DNA strands attached to gold by multiple thiol groups display enhanced stability at high temperature compared to those attached by a single thiol.

Although adenine nucleotides have been described for the control of surface density and conformation of DNA, if the DNA is functionalized with another chemical or physical functional unit including but not limited to ligand, a molecule, a macromolecule, an aptamer, a lectin, an immunoglobulin an antibody, a biomolecule, a solid state particle, a vesicle, or a label, the surface density and distance from the surface of such a functional unit could also be controlled by the method described. Such a functional unit could also be attached to the adenine block via a bifunctional linker molecule other than DNA or directly coupled to the adenine block either before or after immobilization of the adenine block on the surface. A person skilled in the art would understand there is a wide variety of methods to couple a chemical or physical functional unit either directly to an adenine block or to a bifunctional linker molecule other than DNA.

Obviously, many modifications and variations of the present invention are possible in light of the above teachings. It is therefore to be understood that, within the scope of the appended claims, the invention may be practiced otherwise than as specifically described.

What is claimed as new and desired to be protected by Letters Patent of the United States is:

1. A method for attaching nucleic acids or nucleic acid analogs to a surface in a controlled conformation comprising:
providing a surface;
providing an immobilization solution comprising at least one nucleic acid or nucleic acid analog, said nucleic acid comprising a functional sequence and at least one block of adenine nucleotides or adenine nucleotide analogs; and
contacting said immobilization solution to said surface for a period of time sufficient to allow said at least one block of adenine nucleotides or adenine nucleotide analogs to attach to said surface.

2. The method of claim 1 wherein said surface is selected from the group consisting of gold iron, cobalt, nickel, copper, ruthenium, rhodium, palladium, silver, osmium, iridium, platinum and alloys of gold, iron, cobalt, nickel, copper, ruthenium, rhodium, palladium, silver, osmium, iridium and platinum.

3. The method of claim 2 wherein said surface is preferably gold.

4. The method of claim 1 wherein said functional sequence comprises one or more DNA, RNA or nucleic acid analog monomer units.

5. The method of claim 1 wherein said at least one block of adenine nucleotides or adenine nucleotide analogs is located at an end of said nucleic acid.

6. The method of claim 1 wherein said at least one block of adenine nucleotides or adenine nucleotide analogs is positioned in the center of the functional sequence.

7. The method of claim 1 wherein said at least one block of adenine nucleotides or adenine nucleotide analogs is positioned asymmetrically within said functional sequence.

8. The method of claim 1 wherein said at least one block of adenine nucleotides or adenine nucleotide analogs has a length equal to or greater than the length of the longest uninterrupted sequence of adenine nucleobases in said functional sequence.

9. The method of claim 6 wherein the length of the at least one block of adenine nucleotides or adenine nucleotide analogs preferably ranges from about 5 to about 25 adenine nucleotides or adenine nucleotide analogs.

10. The method of claim 1 wherein said functional sequence has a length ranging from about 3 to about 200 nucleotides or nucleotide analogs.

11. The method of claim 1 further comprising chemical or physical functional unit attached to said nucleic acid or nucleic acid analog.

12. The method of claim 11 wherein said chemical or physical functional unit is a ligand, a molecule, a macromolecule, an aptamer, a lectin, an immunoglobulin, an antibody a biomolecule, a solid state particle, a vesicle, or a label.

13. The method of claim 1 further comprising: attaching a moiety with a high affinity and/or high specificity for said surface to an end of said at least one block of adenine nucleobases or adenine nucleobase analogs.

14. The method of claim 13 further comprising:
providing at least one lateral spacer block of adenine nucleotides or adenine nucleotide analogs; and
contacting said immobilization solution and said lateral spacer block to said surface for a period of time sufficient to allow said at least one block of adenine nucleotides or adenine nucleotide analogs and said lateral spacer block to attach to said surface.

15. The method of claim 1, further comprising:
providing at least one lateral spacer block of adenine nucleotides or adenine nucleotide analogs; and
contacting said immobilization solution and said lateral spacer block to said surface for a period of time sufficient to allow said at least one block of adenine nucleotides or adenine nucleotide analogs and said lateral spacer block to attach to said surface.

16. A method for controlling the grafting density of nucleic acid or nucleic acid analog attached to a surface comprising:
providing a surface;
providing an immobilization solution comprising at least one nucleic acid or nucleic acid analog, said nucleic acid or nucleic acid analog comprising a functional sequence and at least one block of adenine nucleotides or adenine nucleotide analogs;
providing at least one lateral spacer block of adenine nucleotides or adenine nucleotide analogs; and
contacting said immobilization solution and said lateral spacer block to said surface for a period of time sufficient to allow said at least one block of adenine nucleotides or adenine nucleotide analogs and said lateral spacer block to attach to said surface.

17. The method of claim 16 wherein said surface is selected from the group consisting of gold, iron, cobalt, nickel, copper, ruthenium, rhodium, palladium, silver, osmium, iridium, platinum and alloys of gold, iron, cobalt, nickel, copper, ruthenium, rhodium, palladium, silver, osmium, iridium and platinum.

18. The method of claim 17 wherein said surface is preferably gold.

19. The method of claim 16 wherein said functional sequence comprises one or more DNA, RNA or nucleic acid analog monomer units.

20. The method of claim 16 wherein said at least on block of adenine nucleotides or adenine nucleotide analogs is located at an end of said nucleic acid.

21. The method of claim 16 wherein said at least one block of adenine nucleotides or adenine nucleotide analogs is positioned in the center of the functional sequence.

22. The method of claim 16 wherein said at least one block of adenine nucleotides or adenine nucleotide analogs is positioned asymmetrically within said functional sequence.

23. The method of claim 16 wherein said at least one block of adenine nucleotides or adenine nucleotide analogs has a length equal to or greater than the length of the longest uninterrupted sequence of adenine nucleobases in said functional sequence.

24. The method of claim 23 wherein the length of the at least one block of adenine nucleotides or adenine nucleotide analogs preferably ranges from about 5 to about 25 adenine nucleotides or adenine nucleotide analogs.

25. The method of claim 16 wherein said functional sequence has a length ranging from about 3 to about 200 nucleotides or nucleotide analogs.

26. The method of claim 16 wherein said at least one lateral spacer block of adenine nucleotides or adenine nucleotide analogs ranges in length from about 5 to about 25 nucleotides or nucleotide analogs.

27. The method of claim 16 wherein said grafting density of said nucleic acid or nucleic acid analog is less than about 10¹³ cm⁻².

28. The method of claim 16 further comprising a chemical or physical functional unit attached to said nucleic acid or nucleic acid analog.

29. The method of claim 28 wherein said chemical or physical functional unit is a ligand, a molecule, a macromolecule, an aptamer, a lectin, an immunoglobulin, an antibody, a biomolecule, a solid state particle, a vesicle, or a label.

30. The method of claim 16 further comprising:
attaching a moiety with a high affinity and/or high specificity for said surface to an end of said at least one block of adenine nucleobases or adenine nucleobase analogs.

31. The method of claim 16 further comprising providing a moiety with a high affinity and/or high specificity for said surface, said moiety being attached to said at least one lateral spacer block of adenine nucleotides or adenine nucleotide analogs.

32. The method of claim 16 further comprising:
attaching a moiety with a high affinity and/or high specificity for said surface to an end of said at least one block of adenine nucleobases or adenine nucleobase analogs; and
providing a second moiety, said second ligand being attached to said at least one lateral spacer block of adenine nucleotides or adenine nucleotide analogs.

33. A method for controlling the conformation of nucleic acid or nucleic acid analog attached to a surface comprising:
providing a surface;
providing an immobilization solution comprising at least one nucleic acid or nucleic acid analog, said nucleic acid or nucleic acid analog comprising a functional sequence and at least one block of adenine nucleotides or adenine nucleotide analogs, wherein said at least one block of adenine nucleotides or adenine nucleotide analogs is positioned within the functional sequence; and
contacting said immobilization solution to said surface for a period of time sufficient to allow said at least one block of adenine nucleotides or adenine nucleotide analogs to attach to said surface.

34. The method of claim 33 wherein said at least one block of adenine nucleotides or adenine nucleotide analog is positioned in the center of the functional sequence.

35. The method of claim 33 wherein said at least one block of adenine nucleotides or adenine nucleotide analog is positioned asymmetrically within said functional sequence.

36. The method of claim 33 wherein said surface is selected from the group consisting of gold, iron, cobalt, nickel, copper, ruthenium, rhodium, palladium, silver, osmium, iridium, platinum and alloys of gold, iron, cobalt, nickel, copper, ruthenium, rhodium, palladium, silver, osmium, iridium and platinum.

37. The method of claim 36 wherein said surface is preferably gold.

38. The method of claim 33 wherein said functional sequence comprises one or more DNA, RNA or nucleic acid analog monomer units.

39. The method of claim 33 wherein said at least one block of adenine nucleotides or adenine nucleotide analogs has a length equal to or greater than the length of the longest uninterrupted sequence of adenine nucleobases in said functional sequence.

40. The method of claim 33 wherein the length of the at least one block of adenine nucleotides or adenine nucleotide analogs preferably ranges from about 5 to about 25 adenine nucleotides or adenine nucleotide analogs.

41. The method of claim 33 wherein said functional sequence has a length ranging from about 3 to about 400 nucleotides or nucleotide analogs.

42. The method of claim 33 further comprising a chemical or physical functional unit attached to said nucleic acid or nucleic acid analog.

43. The method of claim 42 wherein said chemical or physical functional unit is a ligand, a molecule, a macromolecule, an aptamer, a lectin, an immunoglobulin, an antibody, a biomolecule, a solid state particle, a vesicle, or a label.

44. The method of claim 33 further comprising:
providing at least one lateral spacer block of adenine nucleotides or adenine nucleotide analogs; and
contacting said immobilization solution and said lateral spacer block to said surface for a period of time sufficient to allow said at least one block of adenine nucleotides or adenine nucleotide analogs and said lateral spacer block to attach to said surface.

45. A method for controlling the conformation of nucleic acids attached to a surface comprising:
providing a surface;
providing an immobilization solution comprising at least one nucleic acid or nucleic acid analog, said nucleic acid or nucleic acid analog comprising a functional sequence and at least two blocks of adenine nucleotides or adenine nucleotide analogs; and
contacting said immobilization solution to said surface for a period of time sufficient to allow said at least two blocks of adenine nucleotides or adenine nucleotide analogs to attach to said surface.

46. The method of claim 45 wherein said at least two blocks of adenine nucleotides or adenine nucleotide analogs are positioned within the functional sequence.

47. The method of claim 45 wherein said at least two blocks of adenine nucleotides or adenine nucleotide analogs are positioned at the ends of said functional sequence.

48. The method of claim 45 wherein said surface is selected from the group consisting of gold, iron, cobalt, nickel, copper, ruthenium, rhodium, palladium, silver, osmium, iridium, platinum and alloys of gold, iron, cobalt, nickel, copper, ruthenium, rhodium, palladium, silver, osmium, iridium and platinum.

49. The method of claim 48 wherein said surface is preferably gold.

50. The method of claim 45 wherein said functional sequence comprises one or more DNA, RNA or nucleic acid analog monomer units.

51. The method of claim 45 wherein each of said at least two blocks of adenine nucleotides or adenine nucleotide analogs has a length equal to or greater than the length of the longest uninterrupted sequence of adenine nucleobases in said functional sequence.

52. The method of claim 45 wherein the length of each of the at least two blocks of adenine nucleotides or adenine nucleotide analogs preferably ranges from about 5 to about 25 adenine nucleotides.

53. The method of claim 45 wherein said functional sequence has a length ranging from about 3 to about 400 nucleotides or nucleotide analogs.

54. The method of claim 45 further comprising a chemical or physical functional unit attached to said nucleic acid or nucleic acid analog.

55. The method of claim 54 wherein said chemical or physical functional unit is a ligand, a molecule, a macromolecule, an aptamer, a lectin, an immunoglobulin, an antibody, a biomolecule, a solid state particle, a vesicle, or a label.

56. The method of claim 45 further comprising:
providing at least one lateral spacer block of adenine nucleotides or adenine nucleotide analogs; and
contacting said immobilization solution and said lateral spacer block to said surface for a period of time sufficient to allow said at least one block of adenine nucleotides or adenine nucleotide analogs and said lateral spacer block to attach to said surface.

57. A method for attaching chemical or physical functional units to a surface at a controlled surface density comprising:
providing a surface;
providing an solution comprising at least one chemical or physical functional unit and at least one block of adenine nucleotides or adenine nucleotide analogs; and
contacting said solution to said surface for a period of time sufficient to allow said at least one block of adenine nucleotides or adenine nucleotide analogs to attach to said surface.

58. The method of claim 57 wherein said functional unit is a ligand, a molecule, a macromolecule, an aptamer, a lectin, an immunoglobulin, an antibody, a biomolecule, a solid state particle, a vesicle, or a label.

59. The method of claim 57 wherein said surface is selected from the group consisting of gold, iron, cobalt, nickel, copper, ruthenium, rhodium, palladium, silver, osmium, iridium, platinum and alloys of gold, iron, cobalt, nickel, copper, ruthenium, rhodium, palladium, silver, osmium, iridium and platinum.

60. The method of claim 59 wherein said surface is preferably gold.

61. The method of claim 57 wherein said at least one block of adenine nucleotides or adenine nucleotide analogs is attached to said functional unit.

62. The method of claim 61 wherein said at least one block of adenine nucleotides or adenine nucleotide analogs is positioned at an end of said functional unit.

63. The method of claim 61 wherein said at least one block of adenine nucleotides or adenine nucleotide analogs is positioned in the center of the functional unit.

64. The method of claim 61 wherein said at least one block of adenine nucleotides or adenine nucleotide analogs is positioned asymmetrically within said functional unit.

65. The method of claim 61 wherein said functional unit is attached to said at least one block by a bifunctional linker molecule.

66. The method of claim 57 wherein the length of the at least one block of adenine nucleotides or adenine nucleotide analogs preferably ranges from about 5 to about 25 adenine nucleotides or adenine nucleotide analogs.

67. The method of claim 57 further comprising:
providing at least one lateral spacer block of adenine nucleotides or adenine nucleotide analogs; and
contacting said solution and said lateral spacer block to said surface for a period of time sufficient to allow said at least one block of adenine nucleotides or adenine nucleotide analogs and said lateral spacer block to attach to said surface.
